(19) United States
(12) Patent Application Publication
(10) Pub. No.: US 2005/0188474 A1
(43) Pub. Date: Sep. 1, 2005

(54) Cosmetic keratin fibre care or makeup composition

(75) Inventors: Valerie De La Poterie, Le Chatelet En Brie (FR); Therese Daubige, Mousseaux-Les-Bray (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/656,201

(22) Filed: Sep. 8, 2003

Related U.S. Application Data
(60) Provisional application No. 60/417,634, filed on Oct. 11, 2002.

(30) Foreign Application Priority Data
Sep. 6, 2002 (FR) 0211098

(51) Int. Cl. A61K 7/075; A61K 7/13

(52) U.S. Cl. 8/404; 510/119; 8/405

(57) ABSTRACT

Cosmetic keratin fibre care or makeup composition which exhibits a solids content defined by a dry solids extract of more than 47% by weight and a consistency index of less than 1 000 Pa.

The present invention relates to a cosmetic keratin fibre care or makeup composition.

The invention likewise pertains to the use of this composition for making up keratin fibres, especially the eyelashes, eyebrows and hair, and to a method of making up or cosmetically caring for the said fibres.

The makeup method and composition according to the invention are more particularly intended for the keratin fibres, particularly substantially longitudinal keratin fibres, of human beings, such as the eyelashes, eyebrows and hair, including false eyelashes; preferably, the makeup method and composition according to the invention are intended for the eyelashes.

The composition may be a makeup composition, a makeup base also called “base coat”, a composition to be applied over makeup, also called topcoat, or else a keratin fibre cosmetic treatment or care composition. The invention relates more particularly to a composition which may be defined as an eye makeup composition, such as a mascara.

Eye makeup compositions, and especially eyelash makeup compositions, such as mascaras, may be in a variety of forms: for example, in the form of two-phase oil-in-water or O/W emulsions or water-in-oil or W/O emulsions, or aqueous or anhydrous dispersions.

These compositions are characterized by their solids content, which is provided in part by a dispersed fatty phase consisting, for example, of one or more waxes for the purpose of bringing substance onto the eyelashes and hence obtaining a volumizing makeup result.

It is known from the prior art that, the greater the increase in solids content in a composition, the greater the deposition of substance on the eyelash and hence the more volumizing the result obtained will be.

However, increasing the solids content, in other words most often the amount of waxes in a composition, such as an emulsion or dispersion, entails increasing the consistency of the resultant product, with the consequence that application to the eyelashes is complicated and difficult, since the product is thick and viscous: it goes on with difficulty, heterogeneously and in lumps.

The increase in solids content is therefore often limited by the increase in consistency and does not exceed 45% of the total weight of the composition.

This limitation on the solids content is often linked to the impossibility of increasing the wax content in the fatty phase, which does not exceed 25% for reasons of feasibility; and, between 20% and 25% by weight of wax, the compositions are often very thick, compact and difficult to apply and exhibit unsatisfactory cosmetic properties.

Another means of increasing the solids content is to incorporate solid particles such as fillers or pigments, but the increase in consistency again limits the maximum percentage of solids; furthermore, the use of solid particles in large quantity is detrimental to smooth and homogeneous deposition, owing not only to the consistency but also to the size of the particles introduced, giving a rough, granular appearance to the deposition.

This is generally the case with what are termed volumizing mascaras, which are difficult to apply and give a heterogeneous makeup result.

Conversely it is possible to formulate compositions which are of low consistency and are easy to apply; in that case, however, the wax content is low, giving rise to an unsatisfactory makeup effect.

It is therefore not possible to obtain a keratin fibre makeup composition, in particular an “eye makeup” composition, which comprises a high solids content and which exhibits consistency satisfactory for easy and homogeneous application in conjunction with a satisfactory volumizing and separating effect.

There is therefore a need for a cosmetic keratin fibre makeup composition which has a high solids content while exhibiting and retaining a low consistency index.

There is also a need for a cosmetic keratin fibre makeup composition, for example an eye makeup composition, such as a mascara, which exhibits excellent application properties at the time of its application: that is, which allows in particular an easy and homogeneous application and which at the same time also provides excellent results with regard to the final makeup result, in particular a good volumizing effect and a good separating effect.

There is still a need for a composition which allows to obtain a quick make up of keratinic fibers.

The aim of the invention is to provide a cosmetic keratin fibre care or makeup composition which responds inter alia to these needs.

The aim of the invention is also to provide a keratin fibre care or makeup composition, such as a mascara composition, which solves the problems of the prior art compositions and which does not exhibit the drawbacks, limitations, faults and disadvantages of the prior art compositions.

This aim along with others is achieved in accordance with the invention by a cosmetic keratin fibre care or makeup composition which has a solids content defined by a dry solids extract of more than 47% by weight, and a consistency index of less than 1 000 Pa.

The compositions according to the invention, exhibit a combination of a high solids content, specifically of more than 47% by weight, and a low consistency index, specifically of less than 1 000 Pa, which has never been described or suggested in the prior art.

The consistency index is a parameter which makes it possible to take account of the overall consistency of the product.

Owing to the high solids content of the final composition and a satisfactory, low consistency index, generally of the order of that of the known mascaras of the prior art, the surprising result is one of easy application and homogeneous deposition in tandem with a satisfactory separating and volumizing effect.

In other words, the composition according to the invention makes it possible for the first time in consequence of the combination of two specific parameters, each located within a specific zone, to combine excellent application properties of the composition with excellent properties of deposition of the makeup obtained with this composition.

The compositions according to the invention solve the problems of the prior art compositions and do not exhibit the faults, limitations and disadvantages of the prior art compositions.

In particular the compositions of the invention overcome the widespread prejudice in the prior art according to which it was impossible to obtain a makeup composition, in particular an eye makeup composition, which exhibits both a high solids content and a consistency satisfactory for easy and homogeneous application in combination with a satisfactory volumizing and separating effect.

Furthermore, the composition according to the invention is stable, even over a long period of time, and homogeneous. The dry solids extract is advantageously more than 48% by weight and preferably more than 50% by weight. In particular the dry extract of the composition is less than 85% by weight, preferably less than 75% and better still less than 65%.

The higher the solids content, defined by the dry solids extract, the greater the volumizing effect, while application still remains easy and the deposition homogeneous, even at such high solids contents.

The consistency index is advantageously from 1 to 900, preferably from 10 to 800; within these preferred ranges, the application properties are further improved and are always in tandem with excellent volumizing and separating effects.

The composition according to the invention advantageously comprises at least one fatty phase comprising at least one particular, specific structuring agent which may, surprisingly, be incorporated into the composition even in very large amounts, which may range, for example, up to 50% by weight of the composition without substantially increasing the consistency index or causing the composition to solidify.

The total fatty phase of the composition may represent from 10 to 60%, preferably from 15 to 50%, more preferably from 20 to 40% of the total weight of the composition.

The incorporation of a specific structuring agent of this kind in part or in whole into the fatty phase is possible without greatly increasing the consistency index and it is therefore possible, by virtue of these specific structuring agents, to attain solids contents of more than 47%—much higher than in the prior art.

The incorporation of this specific structuring agent into the fatty phase of the composition makes it possible, surprisingly, to obtain a high solids content in combination with a satisfactory consistency and hence to obtain the combination of easy and homogeneous application with a satisfactory volumizing and separating effect.

In other words it has been possible according to the invention to obtain compositions which are stable and homogeneous with a dry solids extract of more than 47% by using a specific structuring agent, while moreover retaining, surprisingly, a consistency index of less than 1 000 Pa.

Another possible advantage of using this specific structuring agent in the compositions of the invention is that it also allows improved attachment to the keratin fibre, such as the eyelash, in a manner which is homogeneous and rapid, owing to its optionally tacky character.

It has been found that the incorporation of such an agent into the fatty phase of the composition of the invention, the said agent being defined by specific values of certain parameters, would precisely make it possible to obtain the combination of high solids contents and desired low consistencies, and so to obtain the combination of desired properties and effects.

The structuring agent or agents may be selected from tacky waxes or combinations of a specific compound with at least one oil, and mixtures thereof.

In this case, the presence of an oil is especially suitable for a cosmetic keratin fibre care or treatment composition, especially for eye lashes.

A first type of structuring agent comprises one or more tackifying waxes.

The said waxes are defined as being “tackifying” waxes.

The wax (or waxes) present in the composition according to the invention generally has(have) a tack greater than or equal to 0.7 N.s, in particular ranging from 0.7 N.s to 30 N.s; preferably greater than or equal to 1 N.s, in particular ranging from 1 N.s to 20 N.s; and more preferably greater than or equal to 2 N.s, in particular ranging from 2 N.s to 10 N.s, and better still from 2 N.s to 5 N.s.

The tackifying wax (or waxes) generally has(have) a hardness of less than or equal to 3.5 MPa, preferably ranging from 0.01 to 3.5 MPa, more preferably ranging from 0.05 MPa to 3 MPa, and better still ranging from 0.1 MPa to 2.5 MPa.

A “wax” for the purposes of the present invention is a lipophilic fatty compound which is solid at ambient temperature (25° C.) and atmospheric pressure (760 mmHg or 10⁵ Pa) which exhibits a reversible solid/liquid change of state, having a melting temperature of more than 30° C. and better still more than 55° C., which can range up to 200° C., in particular up to 120° C.

By taking the wax to its melting temperature it is possible to make it miscible with the oils and to form a microscopically homogeneous mixture, but by taking the temperature of the mixture to ambient temperature the wax recrystallizes in the oils of the mixture.

The melting point values correspond according to the invention to the melting peak measured by means of a differential scanning calorimeter (DSC), for example the calorimeter sold under the name DSC 30 by Mettler, with a temperature rise of 5 or 10° C. per minute.

The tack of the wax is measured at 20° C. using the texturometer sold under the name TA-TX2i by Rheo, equipped with an acrylic polymer traveller in the shape of a cone forming an angle of 45°, by measuring the change in the force (compressive force or stretching force) (F) as a function of the time, during the following operation:

The traveller is moved at a speed of 0.5 mm/s and then penetrates the wax to a depth of 2 mm. When the traveller has penetrated the wax to the depth of 2 mm, the traveller is held fixed for 1 second (corresponding to the relaxation time) and then withdrawn at a speed of 0.5 mm/s. During the relaxation time the force (compressive force) decreases sharply to reach zero and then, during the withdrawal of the traveller, the force (stretching force) becomes negative before again increasing towards the value 0. The tack corresponds to the integral of the curve of force as a function of time for the portion of the curve corresponding to negative values of the force (stretching force). The value of the tack is expressed in N.s.

To carry out the measurement of the tack of the wax, the wax is melted at a temperature equal to the melting point of the wax +10° C. The melted wax is poured into a container 25 mm in diameter and 20 mm in depth. The wax is recrystallized at ambient temperature (25° C.) for 24 hours such that the surface of the wax is flat and smooth, and then the wax is stored for at least 1 hour at 20° C. before the tack measurement is carried out.

The hardness of the wax is measured at 20° C. using the texturometer sold under the name TA-TX2i by Rheo, equipped with a stainless steel traveller in the shape of a cylinder with a diameter of 2 mm, by measuring the change in the force (compressive force or stretching force) (F) as a function of the time during the following operation:

The traveller is moved at a speed of 0.1 mm/s and then penetrates the wax to a depth of 0.3 mm. When the traveller has penetrated the wax to the depth of 0.3 mm, the traveller is held fixed for 1 second (corresponding to the relaxation time) and then withdrawn at a speed of 0.1 mm/s. During the relaxation time the force (compressive force) decreases sharply to reach zero and then, during the withdrawal of the traveller, the force (stretching force) becomes negative before again increasing towards the value 0. The hardness corresponds to the maximum compressive force measured between the surface of the traveller and the wax at the moment they are contacted. The value of this force is expressed in MPa.

To carry out the measurement of the hardness of the wax, the wax is melted at a temperature equal to the melting point of the wax +20° C. The melted wax is poured into a container 30 mm in diameter and 20 mm in depth. The wax is recrystallized at ambient temperature (25° C.) for 24 hours such that the surface of the wax is flat and smooth, and then the wax is stored for at least 1 hour at 20° C. before the hardness measurement is carried out.

As tackifying wax it is possible to use a C₂₀-C₄₀ alkyl (hydroxystearyloxy)stearate (the alkyl group containing 20 to 40 carbon atoms), in particular a C₂₀-C₄₀ alkyl 12-(12′-hydroxystearyloxy)stearate, of formula (I):

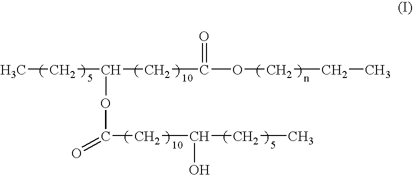

in which n is an integer ranging from 18 to 38, or a mixture of compounds of formula (I).

The invention likewise provides a keratin material care makeup composition comprising in a cosmetically acceptable medium a C₂₀-C₄₀ alkyl (hydroxy-stearyloxy)stearate (in particular a C₂₀-C₄₀ alkyl 12-(12′-hydroxystearyloxy)stearate), especially of formula (I) as described above.

Such a wax is sold in particular under the names Kester Wax K 82 P and Kester Wax K 80 P by Koster Keunen.

A second type of structuring agent comprises a combination of a specific compound with an oil.

This specific compound may be selected from semi-crystalline polymers; fatty-phase Theological agents, such as polyamide-type polymers and hydrophobic silicas; and mixtures thereof.

It is specified that, according to the invention, in the case of the abovementioned combinations, the term “oil” refers to a fatty substance which is liquid at ambient temperature.

It is further specified that the term “volatile compound”, for example “volatile oil”, refers, in the sense of the invention, to any compound (or non-aqueous medium) capable of evaporating in contact with the skin or keratin fibre in less than one hour at ambient temperature and atmospheric pressure. The volatile compound is a volatile cosmetic compound which is liquid at ambient temperature, having in particular a non-zero vapour pressure at ambient temperature and atmospheric pressure, in particular having a vapour pressure ranging from 0.13 Pa to 40 000 Pa (10⁻³ at 300 mmHg), in particular ranging from 1.3 Pa to 13 000 Pa (0.01 to 100 mmHg) and more particularly ranging from 1.3 Pa to 1 300 Pa (0.01 to 10 mmHg).

Conversely a “non-volatile compound”, for example “non-volatile oil”, is a compound remaining on the skin or keratin fibre at ambient temperature and atmospheric pressure for at least several hours and having in particular a vapour pressure of less than 10⁻³ mmHg (0.13 Pa).

The oil may be selected from volatile and non-volatile hydrocarbon and/or silicone and/or fluorinated oils and mixtures thereof. These oils may be animal, vegetable, mineral or synthetic in origin. By “hydrocarbon oil” is meant an oil containing primarily carbon and hydrogen atoms and optionally one or more functional groups selected from hydroxyl, ester, ether and carboxyl functional groups. Possible examples of oils which can be used in the invention include
hydrocarbon oils of animal origin such as perhydrosqualene;
vegetable hydrocarbon oils such as liquid triglycerides of fatty acids of 4 to 24 carbon atoms, such as the triglycerides of heptanoic or octanoic acid or else sunflower oil, maize oil, soya oil, marrow oil, grapeseed oil, sesame oil, hazelnut oil, apricot oil, macadamia oil, castor oil or avocado oil, caprylic/capric acid triglycerides such as those sold by Stearineries Dubois or those sold under the names Miglyol 810, 812 and 818 by Dynamit Nobel, jojoba oil and karite butter;
linear or branched hydrocarbons of mineral or synthetic origin, such as liquid paraffins and their derivatives, vaseline, polydecenes, polybutenes and hydrogenated polyisobutene such as Parleam;
synthetic esters and ethers, particularly those of fatty acids, such as the oils of formula R₁COOR₂ in which R₁ represents the residue of a higher fatty acid containing 1 to 40 carbon atoms and R₂ represents a hydrocarbon chain containing 1 to 40 carbon atoms, with R₁+R₂≧10, such as, for example, purcellin oil, isononyl isononanoate, isopropyl myristate, 2-ethylhexyl palmitate, 2-octyldodecyl stearate, 2-octyldodecyl erucate, isostearyl isostearate, tridecyl trimellitate; hydroxy esters such as isostearyl lactate, octyl hydroxystearate, octyldodecyl hydroxystearate, diisostearyl malate, triisocetyl citrate and fatty alkyl heptanoates, octanoates and decanoates; polyol esters such as propylene glycol dioctanoate, neopentylglycol diheptanoate and diethylene glycol diisononanoate; and pentaerythritol esters such as pentaerythrityl tetraisostearate;
fatty alcohols having 12 to 26 carbon atoms, such as octyldodecanol, 2-butyloctanol, 2-hexyldecanol, 2-undecylpentadecanol and oleyl alcohol;
fluorinated oils optionally partially with hydrocarbon and/or silicone moieties;
silicone oils such as volatile or non-volatile linear or cyclic polydimethylsiloxanes (PDMS); polydimethyl-siloxanes containing alkyl, alkoxy or phenyl groups, pendant or at the end of the silicone chain, groups having 2 to 24 carbon atoms; phenyl silicones such as phenyltrimethicones, phenyldimethicones, phenyltri-methylsiloxydiphenylsiloxanes, diphenyldimethicones, diphenylmethyldiphenyltrisiloxanes and 2-phenylethyl trimethylsiloxysilicates, and
mixtures thereof.

The oil preferably has a molecular mass greater than or equal to 250 g/mol, in particular between 250 and 10 000 g/mol, preferably greater than or equal to 300 g/mol, in particular between 300 and 8 000 g/mol and better still greater than or equal to 400 g/mol, in particular between 400 and 5 000 g/mol.

In the fatty phase, the ratio of the oil(s) to the specific compound(s) is generally from 10/90 to 90/10, preferably from 20/80 to 80/20 and more preferably from 30/70 to 70/30.

This oil may be selected from:
polybutylenes such as Indopol H-100 (of molar mass or MM=965 g/mol), Indopol H-300 (MM=1340 g/mol) and Indopol H-1500 (MM=2160 g/mol), which are sold or manufactured by Amoco;
hydrogenated polyisobutylenes such as Panalane H-300 E, sold or manufactured by Amoco (M=1340 g/mol), Viseal 20000, sold or manufactured by Synteal (MM=6 000 g/mol) and Rewopal PIB 1000, sold or manufactured by Witco (MM=1 000 g/mol);
polydecenes and hydrogenated polydecenes such as Puresyn 10 (MM=723 g/mol) and Puresyn 150 (MM=9 200 g/mol), sold or manufactured by Mobil Chemicals,
esters such as
esters of linear fatty acids having a total carbon number ranging from 30 to 70, such as pentaerythrityl tetrapelargonate (MM=697.05 g/mol),
hydroxy esters such as diisostearyl malate (MM=639 g/mol),
aromatic esters such as tridecyl trimellitate (MM=757.19 g/mol),
esters of C24-C28 branched fatty acids or fatty alcohols, such as those described in EP-A-0 955 039, and especially triisocetyl citrate (MM=865 g/mol), pentaerythrityl tetraisononanoate (MM=697.05 g/mol), glyceryl triisostearate (MM=891.51 g/mol), glyceryl 2-tridecyltetradecanoate (MM=1143.98 g/mol), pentaerythrityl tetraisostearate (MM=1 202.02 g/mol), poly-2-glyceryl tetraisostearate (MM=1 232.04 g/mol) or else pentaerythrityl 2-tetra-decyltetradecanoate (MM=1 538.66 g/mol),
oils of plant origin such as sesame oil (820.6 g/mol), and
mixtures thereof.

In the second embodiment the composition of the invention in which the structuring agent comprises the combination of a specific compound, such as a semi-crystalline polymer, with an oil, the structuring agent may be characterized by:
a tack value ≧0.1 N.s, in particular from 0.1 to 30 N.s; preferably ≧0.5 N.s, in particular from 0.5 N.s to 20 N.s; more preferably ≧0.8 N.s, in particular from 0.8 to 10 N.s; and better still ≧1, in particular from 1 to 5 N.s;
a hardness value ≦30 MPa, in particular between 0.01 to 30 MPa; preferably between 0.05 to 25 MPa; better still between 0.1 and 20 MPa.

More specifically, in relation to the definition of the specific compound in combination with the oil, mentioned earlier on, it is indicated that “polymers” for the purposes of the invention are compounds comprising at least 2 repeating units, preferably at least 3 repeating units and more especially at least 10 repeating units.

In accordance with the invention, the term “semi-crystalline polymer” refers to polymers containing a crystallizable portion, a crystallizable pendant chain or a crystallizable block in the skeleton, and an amorphous portion in the skeleton, and having a first-order reversible phase-change temperature, in particular of fusion (solid-liquid. transition). When the crystallizable portion is in the form of a crystallizable block of the polymeric skeleton, the amorphous portion of the polymer is in the form of an amorphous block; in this case the semi-crystalline polymer is a block copolymer of, for example, diblock, triblock or multiblock type comprising at least one crystallizable block and at least one amorphous block. A block generally comprises at least 5 identical repeating units. The crystallizable block or blocks are then different in chemical nature from the amorphous block or blocks.

The semi-crystalline polymer according to the invention has a melting point of greater than or equal to 30° C. (in particular ranging from 30° C. to 80° C.), preferably ranging from 30° C. to 60° C. This melting point is a first-order change-of-state temperature.

This melting point can be measured by any known method and in particular by means of a differential scanning calorimeter (DSC).

Advantageously the semi-crystalline polymer or polymers to which the invention applies exhibit a number-average molecular mass of greater than or equal to 1 000.

Advantageously the semi-crystalline polymer or polymers of the composition of the invention have a number-average molecular mass \overline{M}n ranging from 2 000 to 800 000, preferably from 3 000 to 500 000, better still from 4 000 to 150 000, and in particular less than 100 000, and better still from 4 000 to 99 000. Preferably they have a number-average molecular mass of more than 5 600, ranging for example from 5 700 to 99 000.

A “crystallizable chain or block” in the sense of the invention is a chain or block which if it were alone would pass reversibly from the amorphous state to the crystalline state depending on whether the temperature was above or below the melting point. A chain in the sense of the invention is a group of atoms which is pendant or lateral relative to the skeleton of the polymer. A block is a group of atoms belonging to the skeleton, the group constituting one of the repeating units of the polymer. Advantageously the “crystallizable pendant chain” may be a chain containing at least 6 carbon atoms.

The crystallizable block(s) or chain(s) of the semi-crystalline polymers preferably represent at least 30% of the total weight of each polymer and better still at least 40%. The semi-crystalline polymers of the invention containing crystallizable blocks are block or multiblock polymers. They can be obtained by polymerizing monomers containing reactive double bonds (or ethylenic bonds) or by polycondensation. When the polymers of the invention are polymers containing crystallizable side chains they are advantageously in random or statistical form.

The semi-crystalline polymers of the invention are preferably synthetic in origin. Moreover, they do not include a polysaccharide skeleton. Generally speaking, the crystallizable units (chains or blocks) of the semi-crystalline polymers according to the invention originate from one or more monomers containing crystallizable block(s) or chain(s) used for the preparation of the semi-crystalline polymers.

In accordance with the invention the semi-crystalline polymer may be selected from block copolymers containing at least one crystallizable block and at least one amorphous block, homopolymers and copolymers carrying at least one crystallizable side chain per repeating unit, and mixtures thereof.

The semi-crystalline polymers which can be used in the invention are in particular:
block polyolefin copolymers of controlled crystallization, particularly those whose monomers are described in EP-A-0 951 897,
polycondensates, particularly those of aliphatic or aromatic polyester type or aliphatic/aromatic copolyester type,
homopolymers or copolymers carrying at least one crystallizable side chain and homopolymers or copolymers carrying in the skeleton at least one crystallizable block, such as those described in U.S. Pat. No. 5,156,911,
homopolymers or copolymers carrying at least one crystallizable side chain, in particular containing one or more fluorine-containing groups, such as those described in WO-A-01/19333,
and mixtures thereof. In these two latter cases the crystallizable side chain(s) or block(s) are hydrophobic.

A) Semi-crystalline polymers having crystallizable side chains

Mention may be made in particular of those defined in U.S. Pat. No. 5,156,911 and WO-A-01/19333. These are homopolymers or copolymers containing from 50 to 100% by weight of units resulting from the polymerization of one or more monomers which carry a crystallizable hydrophobic side chain.

These homopolymers or copolymers are of any kind, provided that they meet the conditions indicated above.

They may result
from the polymerization, in particular the free-radical polymerization, of one or more monomers containing reactive or ethylenic double bond(s) in respect of a polymerization, namely containing a vinylic (meth)acrylic or allylic group;
from the polycondensation of one or more monomers which carry co-reactive groups (carboxylic or sulphonic acid, alcohol, amine or isocyanate), such as, for example, polyesters, polyurethanes, polyethers, polyureas and polyamides.

Generally speaking these polymers are selected in particular from homopolymers and copolymers resulting from the polymerization of at least one crystallizable-chain monomer which can be representable of formula X: 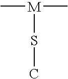
with M representing an atom of the polymeric skeleton, S representing a spacer and C representing a crystallizable group.

The crystallizable chains “-S-C” may be aliphatic or aromatic, optionally fluorinated or perfluorinated. “S” represents in particular a (CH₂)ₙ or (CH₂CH₂O)ₙ or (CH₂O) group which is linear or branched or cyclic, with n being an integer ranging from 0 to 22. “S” is preferably a linear group. Preferably “S” and “C” are different.

When the crystallizable chains “-S-C” are aliphatic hydrocarbon chains they contain alkyl hydrocarbon chains containing at least 11 carbon atoms and not more than 40 carbon atoms, and better still not more than 24 carbon atoms. They are in particular aliphatic chains or alkyl chains possessing at least 12 carbon atoms and preferably the chains in question are C₁₄-C₂₄ alkyl chains. When the chains in question are fluorinated or perfluorinated alkyl chains they contain at least 6 fluorinated carbon atoms and in particular at least 11 carbon atoms of which at least 6 carbon atoms are fluorinated.

Examples of semi-crystalline polymers or copolymers containing one or more crystallizable chains include those resulting from the polymerization of one or more monomers as follows: saturated alkyl (meth)acrylates with the C₁₄-C₂₄ alkyl group, perfluoroalkyl (meth)acrylates with a C₁₁-C₁₅ perfluoroalkyl group, N-alkyl(meth)acrylamides with the C₁₄ to C₂₄ alkyl group with or without a fluorine atom, vinyl esters containing alkyl or perfluoro(alkyl) chains with the C₁₄ to C₂₄ alkyl group (with at least 6 fluorine atoms for a perfluoroalkyl chain, vinyl ethers containing alkyl or perfluoro(alkyl) chains with the C₁₄-C₂₄ alkyl group and at least 6 fluorine atoms for a perfluoroalkyl chain, C₁₄ to C₂₄ alpha-olefins such as, for example, octadecene, para-alkylstyrenes with an alkyl group containing 12 to 24 carbon atoms, and mixtures thereof.

When the polymers result from a polycondensation the hydrocarbon and/or fluorinated crystallizable chains as defined above are carried by a monomer which may be a diacid, a diol, a diamine or a diisocyanate.

When the polymers of the invention are copolymers they also include from 0 to 50% of groups Y or Z resulting from the copolymerization:

α) of Y, which is a polar or non-polar monomer or a mixture of the two:
when Y is a polar monomer it is either a monomer which carries polyoxyalkylenated groups (especially oxyethylenated and/or oxypropylenated), a hydroxyalkyl (meth)acrylate such as hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, a monomer which carries at least one carboxylic acid group such as (meth)acrylic, crotonic, itaconic, maleic or fumaric acid or which carries a carboxylic anhydride group such as maleic anhydride, and mixtures thereof.
When Y is a non-polar monomer it may be an ester of the linear, branched or cyclic alkyl (meth)acrylate type, a vinyl ester, an alkyl vinyl ether, an alpha-olefin, styrene or styrene substituted by a C₁ to C₁₀ alkyl group, such as α-methylstyrene or a polyorganosiloxane-type macromonomer containing vinylic unsaturation.

“Alkyl” for the purposes of the invention is a saturated group, in particular a C₈ to C₂₄ group, unless specifically mentioned, and better still a C₁₄ to C₂₄ group.

β) of Z, which is a polar monomer or a mixture of polar monomers. In this case Z has the same definition as “polar Y” defined above.

Semi-crystalline polymers containing a crystallizable side chain are preferably alkyl (meth)acrylate or alkyl(meth)acrylamide homopolymers with an alkyl group as defined above, and in particular C₁₄-C₂₄ group, copolymers of these monomers with a hydrophilic monomer, preferably of a different kind from (meth)acrylic acid, such as N-vinylpyrrolidone, or hydroxyethyl (meth)acrylate, and mixtures thereof.

B) Polymers carrying in the skeleton at least one crystallizable block

These polymers are, in particular, block copolymers consisting of at least 2 chemically different blocks of which one is crystallizable.
It is possible to use the block polymers defined in patent U.S. Pat. No. 5,156,911;
block copolymers of olefin or of cycloolefin containing a crystallizable chain, such as those obtained from the block polymerization of
cyclobutene, cylohexene, cyclooctene, norbornene (i.e. bicyclo[2.2.1]hept-2-ene), 5-methyl-norbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydro-naphthalene, dicyclopentadiene or mixtures thereof
with ethylene, propylene, 1-butene, 3-methyl-1-butene, 1-hexene, 4-methyl-1-pentene, 1-octene, 1-decene, 1-eicosene or mixtures thereof,
and in particular block copoly(ethylene/norbornene) and block (ethylene/propylene/ethylidene-norbornene) terpolymers. It is also possible to use those resulting from the block copolymerization of at least two C₂-C₁₆ and better still C₂-C₁₂ and even better still C₄-C₁₂ α-olefins, such as those mentioned above, and in particular the block bipolymers of ethylene and of 1-octene.

The copolymers may be copolymers having at least one crystallizable block, the remainder of the copolymer being amorphous (at ambient temperature). These copolymers may further comprise two chemically different crystallizable blocks. Preferred copolymers are those which possess at ambient temperature both a crystallizable block and an amorphous block which is both hydrophobic and lipophilic, the blocks being distributed sequentially; mention may be made, for example, of polymers possessing one of the following crystallizable blocks and one of the following amorphous blocks:
Naturally crystallizable blocks: a) polyesters such as poly(alkylene terephthalate), b) polyolefins such as polyethylenes or polypropylenes.
Amorphous and lipophilic block, such as amorphous polyolefins or copoly(olefin)s such as poly(isobutylene), hydrogenated polybutadiene and hydrogenated poly(isoprene).

Possible examples of such copolymers containing a crystallizable block and an amorphous block, the blocks being distinct, include:

α) block poly(ε-caprolactone)-b-poly(butadiene) copolymers, used preferably in hydrogenated form, such as those described in the article “Melting behavior of poly(ε-caprolactone)-block-polybutadiene copolymers” by S. Nojima, Macromolecules, 32, 3727-3734 (1999).

β) hydrogenated poly(butylene terephthalate)-b-poly(isoprene) block or multiblock copolymers, cited in the article “Study of morphological and mechanical properties of PP/PBT” by B. Boutevin et al., Polymer Bulletin, 34, 117-123 (1995).

γ) the poly(ethylene)-b-copoly(ethylene/-propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997).

δ) the poly(ethylene)-b-poly(ethylethylene) block copolymers cited in the general article “Crystallization in block copolymers” by I. W. Hamley, Advances in Polymer Science, Vol. 148, 113-137 (1999).

The semi-crystalline polymers of the composition of the invention may be non-crosslinked or partially crosslinked, since the degree of crosslinking is not detrimental to their dissolution or dispersion in the liquid fatty phase by heating above their melting point. This may then be a chemical crosslinking, by reaction with a polyfunctional monomer during the polymerization. It may also be a physical crosslinking, which may then be due either to the establishment of bonds of hydrogen type or dipolar type between groups carried by the polymer, such as, for example, the dipolar interactions between carboxylate ionomers, these interactions being low in quantity and borne by the polymer skeleton; or due to phase separation between the crystallizable blocks and the amorphous blocks carried by the polymer.

The semi-crystalline polymers of the composition according to the invention are preferably non-crosslinked.

According to one particular embodiment of the invention the polymer is selected from copolymers resulting from the polymerization of at least one monomer containing a crystallizable chain, selected from saturated C₁₄ to C₂₄ alkyl (meth)acrylates, C₁₁ to C₁₅ perfluoroalkyl (meth)acrylates, C₁₄ to C₂₄ N-alkyl-(meth)acrylamides with or without a fluorine atom, vinyl esters containing C₁₄ to C₂₄ alkyl or perfluoroalkyl chains, vinyl ethers containing C₁₄ to C₂₄ alkyl or perfluoroalkyl chains, C₁₄ to C₂₄ α-olefins, para-alkylstyrenes with an alkyl group containing 12 to 24 carbon atoms, with at least one optionally fluorinated C₁ to C₁₀ monocarboxylic ester or amide, which can be represented by the following formula: 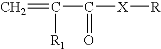
in which R₁ is H or CH₃, R represents an optionally fluorinated C₁-C₁₀ alkyl group and X represents O, NH or NR₂, where R₂ represents an optionally fluorinated C₁-C₁₀ alkyl group.

According to one more particular embodiment of the invention the polymer is derived from a monomer containing a crystallizable chain, selected from saturated C₁₄ to C₂₂ alkyl (meth)acrylates.

As a specific example of a structuring semi-crystalline polymer that can be used in the composition according to the invention, mention may be made of the Interlimer® products from Landec which are described in the brochure “Interlimer® polymers”, Landec IP22 (Rev. 4-97). These polymers are in solid form at ambient temperature (25° C.). They carry crystallizable side chains and have the above formula X.

The semi-crystalline polymers may in particular be:

those described in Examples 3, 4, 5, 7, 9 and 13 of patent U.S. Pat. No. 5,156,911 containing a —COOH group, resulting from the copolymerization of acrylic acid and C₅ to C₁₆ alkyl (meth)acrylate, and more particularly from the copolymerization:
of acrylic acid, hexadecyl acrylate and isodecyl acrylate in a 1/16/3 weight ratio,
of acrylic acid and pentadecyl acrylate in a 1/19 weight ratio,
of acrylic acid, hexadecyl acrylate and ethyl acrylate in a 2.5/76.5/20 weight ratio,
of acrylic acid, hexadecyl acrylate and methyl acrylate in a 5/85/10 weight ratio,
of acrylic acid and octadecyl methacrylate in a 2.5/97.5 weight ratio,
of hexadecyl acrylate, polyethylene glycol methacrylate monomethyl ether containing 8 ethylene glycol units, and acrylic acid in an 8.5/1/0.5 weight ratio.

It is also possible to use the polymer of structure “O” from National Starch, as described in U.S. Pat. No. 5,736,125, with a melting point of 44° C., and also semi-crystalline polymers containing crystallizable pendant chains containing fluorinated groups, as described in Examples 1, 4, 6, 7 and 8 of WO-A-01/19333.

It is additionally possible to use the semi-crystalline polymers obtained by copolymerizing stearyl acrylate and acrylic acid or NVP, as described in U.S. Pat. No. 5,519,063 or EP-A-550745, with melting points of 40° C. and 38° C. respectively.

It is also possible to use semi-crystalline polymers obtained by copolymerizing behenyl acrylate and acrylic acid or NVP, as described in U.S. Pat. No. 5,519,063 and EP-A-550745, with melting points of 60° C. and 58° C. respectively.

The semi-crystalline polymers preferably do not contain a carboxyl group.

The other abovementioned combinations may comprise the combination of a rheological agent with an oil.

This Theological agent is capable of thickening and/or gelling the oil phase. It may be present in an amount which is effective for increasing the viscosity of this phase, particularly until a solid gel is obtained, namely a product which does not flow under its own weight.

This rheological agent is advantageously selected from lipophilic gelling agents, organic gellers and mixtures thereof.

The lipophilic gelling agent may be organic or mineral, polymeric or molecular.

As inorganic lipophilic gelling agent mention may be made of optionally modified clays, such as hectorites modified with a C₁₀ to C₂₂ fatty acid ammonium chloride, such as hectorite modified with distearyl-dimethylammonium chloride.

Mention may also be made of optionally surface-hydrophobicized pyrogenic silica whose particle size is less than 1 μm. It is possible in effect to modify chemically the surface of the silica, by a chemical reaction which brings about a decrease in the number of silanol groups present on the surface of the silica. In particular it is possible to substitute the silanol groups by hydrophobic groups: in that case a hydrophobic silica is obtained. The hydrophobic groups may be:
trimethylsiloxyl groups, obtained in particular by treating pyrogenic silica in the presence of hexamethyldisilazane. Silicas treated in this way are named “silica silylate” according to the CTFA (6th edition, 1995). They are sold, for example, under the names Aerosil R812® by Degussa and CAB-O-SIL TS-530® by Cabot.
dimethylsilyloxyl or polydimethylsiloxane groups, obtained in particular by treating pyrogenic silica in the presence of polydimethylsiloxane or dimethydichlorosilane. Silicas treated in this way are named “silica dimethyl silylate” according to the CTFA (6th edition, 1995). They are sold for example under the names Aerosil R972®, Aerosil R974® by Degussa and CAB-O-SIL TS-610® and CAB-O-SIL TS-720® by Cabot.

The hydrophobic pyrogenic silica preferably has a particle size which can be nanometric to micrometric, ranging for example from 5 to 200 nm approximately.

The polymeric organic lipophilic gelling agents are, for example, partly or completely crosslinked elastomeric organopolysiloxanes of three-dimensional structure, such as those sold under the names KSG6, KSG16 and KSG18 by Shin-Etsu, Trefil E-505C or Trefil E-506C by Dow-Corning, Gransil SR-CYC, SR DMF10, SR-DC556, SR 5CYC gel, SR DMF 10 gel, SR DC 556 gel from Grant Industries, SF 1204 and JK 113 by General Electric; ethylcellulose such as those sold under the name Ethocel by Dow Chemical; polyamides such as copolymers of a C₃₆ diacid condensed with ethylene-diamine, with a weight-average molecular mass of approximately 6000, such as the compounds sold by Arizona Chemical under the names Uniclear 80 and Uniclear 100, gums, especially silicone gums, such as PDMS having a viscosity >100 000 centistokes, galacto-mannans containing from one to six and better still from two to four hydroxyl groups per saccharide unit, substituted by a saturated or unsaturated alkyl chain, such as guar gum alkylated with C₁ to C₆ alkyl chains and better still C₁ to C₃ alkyl chains, and mixtures thereof.

As preferred lipophilic gelling agent use is made of non-polymeric molecular organic gelling agents, also dubbed organic gellers, which are compounds whose molecules are capable of establishing, between themselves, physical interactions leading to self-aggregation of the molecules with formation of a 3D macromolecular network which is responsible for the gelation of the liquid fatty phase.

A “liquid fatty phase” in the sense of the invention is a fatty phase which is liquid at ambient temperature (25° C.) and atmospheric pressure (760 mmHg or 105 Pa) and is composed of one or more fatty substances which are liquid at ambient temperature, also called oils, which are generally mutually compatible.

The super-molecular network may result from the formation of a network of fibrils (owing to stacking or aggregation of organic geller molecules), which immobilizes the molecules of the liquid fatty phase.

The ability to form this network of fibrils, and hence to gel, depends on the nature (or chemical class) of the organic geller, on the nature of the substituents carried by its molecules for a given chemical class, and on the nature of the liquid fatty phase.

The physical interactions are diverse but exclude co-crystallization. These physical interactions are, in particular, interactions such as self-complementary hydrogen interactions, π interactions between unsaturated rings, dipolar interactions, coordination bonds with organometallic derivatives, and combinations thereof. In general, each molecule of an organic geller can establish a number of types of physical interaction with a neighbouring molecule. Thus, advantageously, the molecules of the organic gellers according to the invention include at least one group capable of establishing hydrogen bonds and, better still, at least two groups capable of establishing hydrogen bonds, at least one aromatic ring, and better still at least two aromatic rings, at least one or more bonds with ethylenic unsaturation and/or at least one or more asymmetric carbons. The groups capable of forming hydrogen bonds are preferably selected from hydroxyl, carbonyl, amine, carboxylic acid, amide, urea and benzyl groups and combinations thereof.

The organic geller or gellers according to the invention are soluble in the liquid fatty phase after heating to give a transparent homogeneous liquid phase. They may be solid or liquid at ambient temperature and atmospheric pressure.

The molecular organic geller or gellers which can be used in the composition according to the invention are, in particular, those described in the document “Specialist Surfactants”, edited by D. Robb, 1997, p. 209-263, Chapter 8 by P. Terech, European applications EP-A-1068854 and EP-A-1086945 or else application WO-A-02/47031.

Among these organic gellers mention may be made in particular of the amides of carboxylic acids, especially tricarboxylic acids, such as cyclohexane-tricarboxamides (see European patent application EP-A-1068854), diamides having hydrocarbon chains each containing 1 to 22 carbon atoms, for example 6 to 18 carbon atoms, the said chains being unsubstituted or substituted by at least one substituent selected from ester, urea and fluoro groups (see application EP-A-1086945), and in particular the diamides resulting from the reaction of diaminocyclohexane, in particular diaminocyclohexane in trans form, and an acid chloride such as, for example, N,N′-bis(dodecanoyl)-1,2-diaminocyclohexane, the amides of N-acylamino acids such as the diamides resulting from the action of an N-acylamino acid with amines containing 1 to 22 carbon atoms, such as, for example, those described in WO-93/23008, and especially the amides of N-acylglutamic acid in which the acyl group represents a C₈ to C₂₂ alkyl chain, such as N-lauroyl-L-glutamic acid dibutyl amide, manufactured or sold by Ajinomoto under the name GP-1, and mixtures thereof.

The compositions may contain from 10 to 60% of structuring agent, whether it be of the tackifying wax type or of the combination type or whether it comprise a mixture of the two types. Preferably the composition contains from 15 to 50% by weight, better still from 20 to 40%, of structuring agent, which allows overall solids contents to be attained in the composition of >47%, preferably >48%, and better still >50% while maintaining a satisfactory consistency index <1 000 Pa, preferably between 1 and 900 Pa and better still between 10 and 800 Pa.

Conventional eye makeup compositions, such as mascaras, generally have a consistency index of this order of magnitude but with dry extracts which are less than 47% and often less than 40%; for example, between 30% and 40%; this limits the thickening effect.

One Example A of a Structuring Agent of the Tackifying Wax Type Corresponding to the Invention is the Following:

Commercial name: Kester wax K82P and Kester wax K 80P from Koster Keunen

Tack=3.38 N.s

Hardness=0.96 MPa

One Example B of an Agent Corresponding to the Invention, Consisting of a Semi-Crystalline Polymer in Combination with an Oil, is the Following:

Fatty phase=polybutene(1)/stearyl acrylate-N-vinylpyrrolidone (40/60) copolymer (2) mixture with a melting point of 56° C.
(1): Indopol H 100 from AMOCO
(2): Basic polymer with a melting point of 56° C., prepared in accordance with the following procedure.

A 1 l reactor equipped with a central stirrer with anchor, a condenser and a thermometer is charged with 120 g of cyclohexane, which is heated from the ambient temperature to 80° C. over 45 min. At 80° C., over the course of 2 h, the following mixture C₁ is introduced:
40 g of cyclohexane +4 g of Triganox 141 [2,5-bis(2-ethylhexanoylperoxy)-2,5-dimethylhexane].
30 min after the beginning of the introduction of the mixture C₁, the mixture C₂ is introduced, over 1 h 30 min, this mixture C₂ consisting of:
190 g of stearyl acrylate +10 g of N-vinyl-pyrrolidone +400 g of cyclohexane.

After the two feeds the mixture is left for 3 h more at 80° C. and then all of the cyclohexane present in the reaction mixture is distilled off under atmospheric pressure.

This gives the polymer with an active substance content of 100% by weight.

Its weight-average molecular mass M_{W} is 38 000, expressed in polystyrene equivalents, and its melting point T_{f} is 56° C. as measured by DSC.

Tack=2.63 N.s

Hardness=5.84 MPa

Tack Measurement Protocol

The tack measurement protocol is described below for a wax, although this protocol is also applicable to other structuring agents, especially to structuring agents consisting of the combination of a specific compound and an oil.

The tack of the wax is measured at 20° C. using a texturometer sold under the name TA-TX2i by Rheo, equipped with an acrylic polymer traveller in the shape of a cone forming an angle of 45°, by measuring the change in the force (compressive force or stretching force) (F) as a function of the time during the following operation:

The traveller is moved at a speed of 0.5 mm/s and then penetrates the wax to a depth of 2 mm. When the traveller has penetrated the wax to the depth of 2 mm, the traveller is held fixed for 1 second (corresponding to the relaxation time) and then withdrawn at a speed of 0.5 mm/s. During the relaxation time the force (compressive force) decreases sharply to reach zero and then, during the withdrawal of the traveller, the force (stretching force) becomes negative before again increasing towards the value 0. The tack corresponds to the integral of the curve of force as a function of time for the portion of the curve corresponding to negative values of the force (stretching force). The value of the tack is expressed in N.s.

To carry out the measurement of the tack of the wax, the wax is melted at a temperature equal to the melting point of the wax +10° C. The melted wax is poured into a container 25 mm in diameter and 20 mm in depth. The wax is recrystallized at ambient temperature (25° C.) for 24 hours such that the surface of the wax is flat and smooth, and then the wax is stored for at least 1 hour at 20° C. before the tack measurement is carried out.

Hardness Measurement Protocol

The hardness measurement protocol is described below for a wax, although this protocol is also applicable to other structuring agents, especially to structuring agents consisting of the combination of a specific compound and an oil.

The hardness of the wax is measured at 20° C. using a texturometer sold under the name TA-TX2i by Rheo, equipped with a stainless steel traveller in the shape of a cylinder with a diameter of 2 mm, by measuring the change in the force (compressive force or stretching force) (F) as a function of the time during the following operation:

The traveller is moved at a speed of 0.1 mm/s and then penetrates the wax to a depth of 0.3 mm. When the traveller has penetrated the wax to the depth of 0.3 mm, the traveller is held fixed for 1 second (corresponding to the relaxation time) and then withdrawn at a speed of 0.1 mm/s. During the relaxation time the force (compressive force) decreases sharply to reach zero and then, during the withdrawal of the traveller, the force (stretching force) becomes negative before again increasing towards the value 0. The hardness corresponds to the maximum compressive force measured between the surface of the traveller and the wax at the moment they are contacted. The value of this force is expressed in MPa.

To carry out the measurement of the hardess of the wax, the wax is melted at a temperature equal to the melting point of the wax +20° C. The melted wax is poured into a container 30 mm in diameter and 20 mm in depth. The wax is recrystallized at ambient temperature (25° C.) for 24 hours such that the surface of the wax is flat and smooth, and then the wax is stored for at least 1 hour at 20° C. before the hardness measurement is carried out.

Consistency Index Measurement Protocol

The compositions according to the invention are characterized with the aid of the texture analyser offered under the name TA-TX2i by Rheo.

The test consists in contacting a stainless steel cylindrical probe 12 mm in diameter with the product (a cylindrical container of diameter=35 mm and depth=15 mm filled with product, whose surface is levelled so as to obtain a thoroughly planar surface).

Measurement is repeated three times per product.

The consistency index is measured during the contact phase of the probe with the product. This phase takes place with a constant displacement of 0.2 mm. The probe therefore sinks down into the product down to this depth of 0.2 mm at a rate of 10 mm/s. The force (or stress) measured at this point in time gives the consistency index (in Pa) of the product. The probe is then held in this position for one second.

Solids Content Measurement Protocol

This consists of a measurement of the dry extract of the mascara liquor, which is carried out on a Mettler Toledo HG 53 balance (halogen moisture analyser).

A sample of mascara (2-3 g) is deposited on an aluminium dish and subjected to a temperature of 120° C. for 60 minutes. The measurement of the dry extract corresponds to the monitoring of the mass of the sample over time. The final solids content is therefore the percentage of the final mass (after 60 min) in relation to the initial mass: DE=(final mass/initial mass)×100.

The composition according to the invention is a makeup composition, a makeup base, a “topcoat” composition to be applied over makeup, or a keratin fibre cosmetic treatment or care composition.

The composition according to the invention is applied more particularly to the eyelashes. Consequently the composition of the invention may be a composition for coating the eyelashes, in particular a composition for making up the eyelashes, also called mascara, a composition to be applied over eyelash makeup, also called topcoat, or else a composition for treating the eyelashes, in particular the eyelashes of human beings or false eyelashes. More especially the composition is a mascara.

The composition according to the invention forms a physiologically acceptable medium.

In the present specification a physiologically acceptable medium is a non-toxic medium which is compatible with the keratin fibres of human beings, in particular the eyelashes or eyebrows, such as a cosmetic medium, the cosmetic medium being either a hydrophilic or a lipophilic cosmetic medium.

The composition according to the invention may further comprise an additional wax, different from the tackifying wax described above.

The additional wax may be selected, for example, from beeswax, paraffin waxes, hydrogenated castor oil and silicone waxes.

The wax or waxes (the tackifying wax and/or the additional wax or waxes) present in the composition may be dispersed in particle form in an aqueous medium. In particular the waxes may be present in the form of a wax-in-water emulsion.

The waxes (the tackifying wax and/or the additional wax or waxes) may also be in the form of an aqueous microdispersion of wax particles. An aqueous wax microdispersion is an aqueous dispersion of wax particles in which the size of the said wax particles is less than or equal to approximately 1 μm.

Wax microdispersions are stable dispersions of colloidal wax particles and are described in particular in “Microemulsions Theory and Practice”, L. M. Prince Ed., Academic Press (1977) pages 21-32.

In particular, these wax microdispersions may be obtained by melting the wax in the presence of a surfactant, and optionally a portion of the water, then gradually adding hot water with stirring. Intermediate formation of an emulsion of the water-in-oil type is observed, followed by a phase inversion with, finally, a microemulsion of the oil-in-water type being obtained. On cooling, a stable microdispersion of solid, colloidal wax particles is obtained.

The wax microdispersions may also be obtained by stirring the mixture of wax, surfactant and water with the aid of stirring means such as ultrasound, a high-pressure homogenizer or turbines.

The particles of the wax microdispersion preferably have average sizes of less than 1 μm (in particular ranging from 0.02 μm to 0.99 μm), preferably less than 0.5 μm (in particular ranging from 0.06 μm to 0.5 μm).

These particles are composed essentially of a wax or of a mixture of waxes. They may, however, comprise a minority proportion of oily and/or pastelike fatty additives, a surfactant and/or a customary fat-soluble additive/active agent.

The additional wax may be present in the composition according to the invention in an amount ranging from 0.1% to 50% by weight, relative to the total weight of the composition, preferably from 0.5% to 30% by weight, better still from 1% to 20% by weight.

The composition according to the invention may comprise at least one fatty compound which is pastelike at ambient temperature. A pastelike fatty substance in the sense of the invention is a fatty substance having a melting point ranging from 20 to 55° C., preferably from 25 to 45° C., and/or a viscosity at 40° C. ranging from 0.1 to 40 Pa.s (1 to 400 poises), preferably 0.5 to 25 Pa.s, measured on the Contraves TV or Rhéomat 80, equipped with a rotor rotating at 60 Hz. The skilled worker is able to select the rotor which allows the viscosity to be measured, from the rotors MS-r3 and MS-r4, on the basis of his or her general knowledge, so as to be able to carry out measurement on the pastelike test compound.

These fatty substances are preferably hydrocarbon compounds, optionally of polymeric type; they may also be selected from silicone compounds; they may also be in the form of a mixture of hydrocarbon compounds and/or silicone compounds. In the case of a mixture of different pastelike fatty substances it is preferred to use pastelike hydrocarbon compounds (containing primarily carbon atoms and hydrogen atoms and optionally ester groups) in majority proportion.

Among the pastelike compounds which can be used in the composition according to the invention, mention may be made of lanolins and lanolin derivatives such as acetylated lanolins or oxypropylenated lanolins or isopropyl lanolate having a viscosity of 18 to 21 Pa.s, preferably 19 to 20.5 Pa.s, and/or a melting point of 30 to 55° C., and mixtures thereof. It is also possible to use esters of fatty alcohols or acids, particularly those having 20 to 65 carbon atoms (melting point of the order of 20 to 35° C. and/or viscosity at 40° C. ranging from 0.1 to 40 Pa.s) such as triisostearyl or cetyl citrate; arachidyl propionate; polyvinyl laurate; cholesterol esters such as triglycerides of plant origin, such as hydrogenated vegetable oils, viscous polyesters such as poly(12-hydroxystearic) acid, and mixtures thereof.

Mention may also be made of silicone pastelike fatty substances such as polydimethylsiloxanes (PDMS) having pendant chains of the alkyl or alkoxy type having 8 to 24 carbon atoms, and a melting point of 20-55° C., such as stearyldimethicones, especially those sold by Dow Corning under the trade names DC2503 and DC25514, and mixtures thereof.

The pastelike fatty substance may be present in the composition according to the invention in an amount ranging from 0.01 to 60% by weight relative to the total weight of the composition, preferably ranging from 0.5 to 45% by weight, and better still ranging from 2% to 30% by weight, in the composition.

The composition according to the invention may comprise emulsifying surfactants, present in particular in a proportion ranging from 2 to 30% by weight relative to the total weight of the composition, and better still from 5% to 15%. These surfactants may be selected from anionic surfactants and non-ionic surfactants. Reference may be made to Encyclopedia of Chemical Technology, Kirk-Othmer, Volume 22, pp. 333-432, 3rd edition, 1979, Wiley, for the definition of the properties and functions (emulsifier) of the surfactants, in particular to pp. 347-377 of this reference, for anionic and non-ionic surfactants.

The surfactants used preferentially in the composition according to the invention are selected:
from non-ionic surfactants: fatty acids, fatty alcohols, polyethoxylated or polyglycerolated fatty alcohols such as polyethoxylated stearyl or cetylstearyl alcohols, esters of fatty acid and sucrose, esters of alkyl glucose, in particular polyoxyethylenated fatty C₁-C₆ alkyl glucose esters, and mixtures thereof.
from anionic surfactants: C₁₆-C₃₀ fatty acids neutralized with amines, aqueous ammonia or alkali metal salts, and mixtures thereof.

Preference is given to using surfactants which allow an oil-in-water or wax-in-water emulsion to be obtained.

The composition according to the invention may comprise at least one film-forming polymer.

The film-forming polymer may be present in the composition according to the invention in a solids content ranging from 0.1% to 60% by weight relative to the total weight of the composition, preferably from 0.5% to 40% by weight, and better still from 1% to 30% by weight.

In the present specification the term “film-forming polymer” refers to a polymer which is capable, by itself or in the presence of an auxiliary film-forming agent, of forming a continuous and adherent film on a support, in particular on keratin materials such as the eyelashes.

The film-forming polymer or polymers which may be present in the composition of the invention are different from the “semi-crystalline polymer” as defined earlier on.

Preferably, the film-forming polymer does not comprise crystallisable moieties. If the film-forming polymer comprises crystallisable moieties, said moieties would represent less than 30% by weight of the total weight of the polymer.

Among the film-forming polymers which may be used in the composition of the present invention, mention may be made of synthetic polymers, of free-radical type or of polycondensate type, and of polymers of natural origin, and mixtures thereof.

A free-radical film-forming polymer is a polymer obtained by polymerizing monomers containing unsaturation, in particular ethylenic unsaturation, each monomer being capable of undergoing homopolymerization (unlike polycondensates).

The film-forming polymers of free-radical type may in particular be vinyl polymers or copolymers, especially acrylic polymers.

The vinyl film-forming polymers may result from the polymerization of monomers containing ethylenic unsaturation and having at least one acidic group and/or of esters of these acidic monomers and/or of amides of these acidic monomers.

As acid-group-bearing monomer it is possible to use α,β-ethylenic unsaturated carboxylic acids such as acrylic acid, methacrylic acid, crotonic acid, maleic acid and itaconic acid. It is preferred to use (meth)-acrylic acid and crotonic acid, and more preferably (meth)acrylic acid.

The esters of acidic monomers are advantageously selected from esters of (meth)acrylic acid (also called (meth)acrylates), in particular alkyl (meth)acrylates, especially C₁-C₃₀, preferably C₁-C₂₀, alkyl (meth)acrylates, aryl (meth)acrylates, especially C₆-C₁₀ aryl (meth)acrylates, and hydroxyalkyl (meth)-acrylates, in particular C₂-C₆ hydroxyalkyl (meth)-acrylates.

Among alkyl (meth)acrylates mention may be made of methyl methacrylate, ethyl methacrylate, butyl methacrylate, isobutyl methacrylate, 2-ethylhexyl meth-acrylate, lauryl methacrylate and cyclohexyl methacrylate.

Among hydroxyalkyl (meth)acrylates mention may be made of hydroxyethyl acrylate, 2-hydroxypropyl acrylate, hydroxyethyl methacrylate, and 2-hydroxypropyl methacrylate.

Among aryl (meth)acrylates mention may be made of benzyl acrylate and phenyl acrylate.

Particularly preferred esters of (meth)acrylic acid are alkyl (meth)acrylates.

According to the present invention the alkyl group of the esters can be either fluorinated or perfluorinated: in other words, some or all of the hydrogen atoms of the alkyl group are substituted by fluorine atoms.

Possible examples of amides of acidic monomers include (meth)acrylamides, and especially N-alkyl-(meth)acrylamides, particularly where the alkyl is C₂-C₁₂. Among N-alkyl(meth)acrylamides mention may be made of N-ethylacrylamide, N-t-butylacrylamide, N-t-octylacrylamide and N-undecylacrylamide.

The vinyl film-forming polymers may also result from the homopolymerization or copolymerization of monomers selected from vinyl esters and styrenic monomers. In particular these monomers can be polymerized with acidic monomers and/or their esters and/or their amides, such as those mentioned hitherto.

Possible examples of vinyl esters include vinyl acetate, vinyl neodecanoate, vinyl pivalate, vinyl benzoate and vinyl t-butylbenzoate.

Styrenic monomers include styrene and alpha-methylstyrene.

Film-forming polycondensates include polyurethanes, polyesters, polyester amides, polyamides, epoxy ester resins and polyureas.

Polyurethanes may be selected from anionic, cationic, non-ionic or amphoteric polyurethanes, acrylic polyurethanes, polyurethane-polyvinyl-pyrrolidones, polyester-polyurethanes, polyether-poly-urethanes, polyureas, polyurea-polyurethanes, and mixtures thereof.

Polyesters may be obtained conventionally by polycondensation of dicarboxylic acids with polyols, especially diols.

The dicarboxylic acid may be aliphatic, alicyclic or aromatic. Possible examples of such acids include oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioc acid, 1,3-cyclohexanedicarboxylic acid, 1,4-cyclohexane-dicarboxylic acid, isophthalic acid, terephthalic acid, 2,5-norbornanedicarboxylic acid, diglycolic acid, thiodipropionic acid, 2,5-naphthalenedicarboxylic acid, 2,6-naphthalenedicarboxylic acid. These dicarboxylic acid monomers may be used alone or in a combination of at least two dicarboxylic acid monomers. Among these monomers it is preferred to select phthalic acid, isophthalic acid and/or terephthalic acid.

The diol may be selected from aliphatic, alicyclic and aromatic diols. It is preferred to use a diol selected from ethylene glycol, diethylene glycol, triethylene glycol, 1,3-propanediol, cyclohexane dimethanol and 1,4-butanediol. As other polyols it is possible to use glycerol, pentaerythritol, sorbitol and trimethylolpropane.

The polyester amides may be obtained in a similar way to the polyesters, by polycondensation of diacids with diamines or amino alcohols. As a diamine it is possible to use ethylenediamine, hexamethylene-diamine, meta- or para-phenylenediamine. As an amino alcohol it is possible to use monoethanolamine.

The polyester may further comprise at least one monomer which carries at least one group —SO₃M, with M representing a hydrogen atom, an ammonium ion NH₄⁺ or a metal ion, such as, for example, an Na⁺, Li⁺, K⁺, Mg²⁺, Ca²⁺, Cu²⁺, Fe²⁺ or Fe³⁺ ion. It is possible to use in particular a difunctional aromatic monomer containing such a group —SO₃M.

The aromatic nucleus of the difunctional aromatic monomer additionally carrying a group —SO₃M as described above may be selected, for example, from benzene, naphthalene, anthracene, biphenyl, oxy-biphenyl, sulphonylbiphenyl and methylenebiphenyl nuclei. As examples of a difunctional aromatic monomer further carrying a group —SO₃M, mention may be made of the following: sulphoisophthalic acid, sulpho-terephthalic acid, sulphophthalic acid and 4-sulpho-naphthalene-2,7-dicarboxylic acid.

Preference is given to using copolymers based on isophthalate/sulphoisophthalate, and more particularly copolymers obtained by condensing diethylene glycol, cyclohexanedimethanol, isophthalic acid and/or sulphoisophthalic acid.

The optionally modified polymers of natural origin may be selected from shellac resin, gum sandarac, dammars, elemis, copals, cellulosic polymers, and mixtures thereof.

In accordance with a first embodiment of the composition according to the invention, the film-forming polymer may be a water-soluble polymer and may be present in an aqueous phase of the composition; the polymer is therefore solubilized in the aqueous phase of the composition. Possible examples of water-soluble film-forming polymers include:
proteins such as proteins of plant origin, such as wheat proteins and soya proteins; proteins of animal origin such as keratins, for example keratin hydrolysates and sulphonic keratins;
cellulose polymers such as hydroxyethyl-cellulose, hydroxypropylcellulose, methylcellulose, ethylhydroxyethylcellulose and carboxymethylcellulose, and quaternized cellulose derivatives;
acrylic polymers or copolymers, such as polyacrylates or polymethacrylates;
vinyl polymers, such as polyvinyl-pyrrolidones, copolymers of methyl vinyl ether and malic anhydride, the copolymer of vinyl acetate and crotonic acid, copolymers of vinylpyrrolidone and vinyl acetate; copolymers of vinylpyrrolidone and caprolactam; and polyvinyl alcohol;
polymers of natural origin, optionally modified, such as:
gums arabic, guar gum, xanthan derivatives and karaya gum;
alginates and carragheenans;
glycoaminoglycans and hyaluronic acid and its derivatives;
shellac resin, gum sandarac, dammars, elemis and copals;
deoxyribonucleic acid;
mucopolysaccharides such as chondroitin sulphates,
and mixtures thereof.

In accordance with another variant embodiment of the composition according to the invention, the film-forming polymer may be a polymer which is solubilized in a liquid fatty phase comprising oils or organic solvents such as those described hitherto (in which case the film-forming polymer is referred to as a fat-soluble polymer). A “liquid fatty phase” for the purposes of the invention is a fatty phase which is liquid at ambient temperature (25° C.) and atmospheric pressure (760 mmHg or 10⁵ Pa), which is composed of one or more fatty substances which are liquid at ambient temperature, also called oils, which are generally compatible with one another.

The liquid fatty phase preferably comprises a volatile oil, optionally in a mixture with a non-volatile oil, the oils being selectable from the abovementioned oils.

Possible examples of fat-soluble polymers include vinyl ester copolymers (the vinyl group being connected directly to the oxygen atom of the ester group and the vinyl ester having a saturated, linear or branched hydrocarbon radical of 1 to 19 carbon atoms which is linked to the carbonyl of the ester group) with at least one other monomer which may be a vinyl ester (other than the vinyl ester already present) , an α-olefin (having 8 to 28 carbon atoms), an alkyl vinyl ether (in which the alkyl group contains 2 to 18 carbon atoms) or an allyl or methallyl ester (having a saturated, linear or branched hydrocarbon radical of 1 to 19 carbon atoms which is linked to the carbonyl of the ester group).

These copolymers may be crosslinked by means of crosslinkers which can be either of vinyl type or of allyl or methallyl type, such as tetraallyloxyethane, divinylbenzene, divinyl octanedioate, divinyl dodecane-dioate and divinyl octadecanedioate.

As examples of these copolymers mention may be made of the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl-propionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene crosslinked with 0.2% of divinylbenzene, and allyl propionate/allyl stearate crosslinked with 0.2% of divinylbenzene.

Fat-soluble film-forming polymers also include fat-soluble copolymers, and in particular those resulting from the copolymerization of vinyl esters having 9 to 22 carbon atoms or alkyl acrylates or methacrylates, the alkyl radicals having 10 to 20 carbon atoms.

Fat-soluble copolymers of this kind may be selected from polyvinyl stearate copolymers, polyvinyl stearate copolymers crosslinked using divinylbenzene, diallyl ether or diallyl phthalate, polystearyl (meth)acrylate copolymers, polyvinyl laurate copolymers, polylauryl (meth)acrylate copolymers, it being possible for these poly (meth)acrylates to be crosslinked using ethylene glycol dimethacrylate or tetraethylene glycol dimethacrylate.

The fat-soluble copolymers defined above are known and are described in particular in the application FR-A-2232303; they can have a weight-average molecular weight ranging from 2000 to 500 000 and preferably from 4000 to 200 000.

As fat-soluble film-forming polymers which can be used in the invention, mention may also be made of polyalkylenes and especially the copolymers of C₂-C₂₀ alkenes, such as polybutene, alkylcelluloses with a linear or branched, saturated or unsaturated C₁ to C₈ alkyl radical such as ethylcellulose and propylcellulose, vinylpyrrolidone (VP) copolymers and especially the copolymers of vinylpyrrolidone and of C₂ to C₄₀ or, better still, C₃ to C₂₀ alkene. Possible examples of VP copolymers which can be used in the invention include VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene and VP/acrylic acid/lauryl methacrylate copolymer.

In accordance with another variant embodiment of the composition of the invention, the film-forming polymer may also be present in the composition in the form of particles in dispersion in an aqueous phase or in a non-aqueous solvent phase, known generally under the name latex or pseudolatex. The techniques for preparing these dispersions are well known to the person skilled in the art.

As aqueous dispersions of film-forming polymer it is possible to use the acrylic dispersions sold under the names Neocryl XK-90®, Neocryl A-1070®, Neocryl A-1090®, Neocryl BT-62®, Neocryl A-1079® and Neocryl A-523® by Avecia-Neoresins, Dow Latex 432® by Dow Chemical, Daitosol 5000 AD® by Daito Kasei Kogyo; or else aqueous polyurethane dispersions sold under the names Neorez R-981® and Neorez R-974® by Avecia-Neoresins, Avalure UR-405®, Avalure UR-410®, Avalure UR-425®, Avalure UR-450®, Sancure 875®, Sancure 861®, Sancure 878® and Sancure 2060® by Goodrich, Impranil 85® by the company Bayer, and Aquamere H-1511® by Hydromer; the sulpho polyesters sold under the brand name Eastman AQ® by Eastman Chemical Products, and the vinyl dispersions such as Mexomere PAM and also the acrylic dispersions in isododecane such as Mexomere PAP by Chimex.

The composition according to the invention may comprise a plasticizer which promotes the formation of a film with the film-forming polymer. Such a plasticizer may be selected from all compounds known to the person skilled in the art as being capable of fulfilling the desired function.

The composition according to the invention may further comprise a colorant such as pulverulent colorants, fat-soluble colorants and water-soluble colorants. This colorant may be present in an amount ranging from 0.01% to 30% by weight, relative to the total weight of the composition.

The pulverulent colorants may be selected from pigments and nacres.

The pigments can be white or coloured, mineral and/or organic, and coated or uncoated. The mineral pigments include titanium dioxide, optionally surface-treated, zirconium oxide, zinc oxide or cerium oxide, and also iron oxides or chromium oxides, manganese violet, ultramarine blue, chromium hydrate and ferric blue. The organic pigments include carbon black, pigments of D & C type, and lakes based on cochineal carmine, barium, strontium, calcium and aluminium.

The nacres may be selected from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the type mentioned above, and nacreous pigments based on bismuth oxychloride.

Fat-soluble dyes are, for example, Sudan Red, D&C Red 17, D&C Green 6, β-carotene, soya oil, Sudan Brown, D&C Yellow 11, D&C Violet 2, D&C Orange 5, quinoline yellow and annatto. Water-soluble dyes are, for example, beetroot juice, methylene blue, the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophyll.

The composition of the invention may further comprise any additive commonly used in cosmetology, such as antioxidants, fillers, preservatives, perfumes, neutralizing agents, thickeners, vitamins and mixtures thereof.

According to one particular embodiment of the invention the composition contains no UV filter (organic filter or mineral filter; filter which absorbs or reflects ultraviolet radiation).

The person skilled in the art will of course take care to select any complementary additives and/or their amount such that the advantageous properties of the composition according to the invention are not, or not substantially, adversely affected by the intended addition.

The composition according to the invention may be manufactured by the known processes which are generally used within the cosmetics field.

The composition may comprise water and optionally one or more hydrophilic organic solvents, in other words one or more water-miscible organic solvents, such as alcohols and especially monoalcohols having 2 to 5 carbon atoms, for instance ethanol, isopropanol or n-propanol, polyols having 2 to 8 carbon atoms, for instance glycerol, diglycerol, propylene glycol, ethylene glycol, 1,3-butylene glycol, sorbitol, pentylene glycol, C₃-C₄ ketones and C₂-C₄ aldehydes.

The water or the mixture of water and hydrophilic organic solvent(s) may be present in the composition according to the invention in an amount ranging from 0.1% to 90% by weight, relative to the total weight of the composition, and preferably from 0.1% to 60% by weight.

According to another embodiment, the fatty phase as defined hereinabove may form a continuous phase of the composition.

In particular the composition according to the invention may be anhydrous.

The invention likewise relates to a cosmetic method of treating or making up keratin fibres which comprises applying to the said keratin fibres the composition as described earlier on.

The invention likewise pertains to a method of coating the eyelashes, which comprises applying to the eyelashes the composition described above.

The invention also relates to the use of the composition as described earlier on for making up keratin fibres, to the use of said composition to obtain a quick make-up of keratin fibres, and to the use of this composition for obtaining easy and homogeneous application and makeup which exhibits an excellent volumizing effect.

The invention will now be described with reference to the following examples, which are given by way of illustration and are not limitative.

### EXAMPLES

A number of mascara compositions were produced and characterized according to the invention.

The formulations according to the invention are formulations of the fatty phase/water emulsion type which are defined generally as follows:

| | |
|---|---|
| Fatty phase | x% |
| Stearic acid | 5.82% |
| Neutralizing agents | 2.9% |
| Black iron oxide | y% |
| Hydroxyethylcellulose | 0.91% |
| Gum arabic | 3.45% |
| Additives, preservatives, water | qs |

Described in the table below in Example 1 to 3 are compositions according to the invention, whose solids content and consistency index were measured.

Likewise indicated in this table for comparison are the solids contents and consistency indices of 3 prior art compositions.

TABLE

| | | | | | % of |
|---|---|---|---|---|---|
| | | Consis- | | % of | black |
| | Solids | tency | | fatty | iron |
| | content | index | Type of fatty | phase = | oxide = |
| Test | (%) | (Pa) | phase | x | y |
| No. 1 | 49.6 | 505 | Tackifying wax | 32 | 5.45 |
| according | | | | | |
| to the | | | | | |
| invention | | | | | |
| No. 2 | 52.4 | 953 | Tackifying wax | 35 | 5.45 |
| according | | | (described | | |
| to the | | | above) | | |
| invention | | | | | |
| No. 3 | 56.8 | 490 | Semi-crystalline | 35 | 8 |
| according | | | polymer (EXAMPLE | | |
| to the | | | A above) + oil | | |
| invention | | | (described above - | | |
| | | | EXAMPLE B) | | |
| Compara- | 39 | 2 030 | / | | |
| tive Volum | | | | | |
| Express | | | | | |
| Compara- | 41.3 | 570 | / | / | / |
| tive | | | | | |
| Intencils | | | | | |
| Compara- | 52.7 | 1 280 | / | / | / |
| tive Effet | | | | | |
| Faux Cils | | | | | |

Table

The mascara Volum Express is sold commercially under the Maybelline brand as a volumizing mascara.

The mascara Intencils is sold commercially under the Lancôme brand as a volumizing mascara.

The mascara Effet Faux Cils is sold commercially under the Yves Saint Laurent brand as a volumizing mascara.

The table above shows that only the compositions of the invention exhibit a high or even very high solids content in tandem with a low consistency.

They make it possible to make up the eyelashes rapidly and homogeneously, in a non-granular fashion, and exhibit a good thickening and separating effect.

1-35. (canceled)

36. A composition comprising a solids content defined by a dry solids extract of more than 47% by weight, and having a consistency index of less than 1 000 Pa.

37. The composition according to claim 36, having a dry solids extract of more than 50% by weight.

38. The composition according to claim 36, having a consistency index of from 10 to 800.

39. The composition according to claim 36, further comprising at least one fatty phase comprising at least one structuring agent.

40. The composition according to claim 39, wherein the fatty phase represents from 10 to 60% of the total weight of the composition.

41. The composition according to claim 39, wherein the structuring agent is selected from the group consisting of tackifying waxes, combinations of at least one specific compound with at least one oil, and mixtures thereof.

42. The composition according to claim 41, wherein the structuring agent consists of one or more tackifying waxes.

43. The composition according to claim 42, wherein the waxes have a tack from 2 N.s to 10 N.s.

44. The composition according claim 42, wherein the waxes have a hardness of from 0.05 MPa to 3 MPa.

45. The composition according to claim 41, wherein the structuring agent is a combination of at least one specific compound and at least one oil.

46. The composition according to claim 45, wherein the at least one specific compound is selected from the group consisting of semi-crystalline polymers; fatty-phase Theological agents; and mixtures thereof.

47. The composition according to claim 45, wherein the at least one oil is selected from the group consisting of volatile and non-volatile hydrocarbon oils, silicone oils, fluorinated oils and mixtures thereof.

48. The composition according to any one of claim 45, wherein the oil has a molecular mass greater than or equal to 250 g/mol.

49. The composition according to 45, wherein, in the fatty phase, the ratio of the oil(s) to the specific compound(s) is from 10/90 to 90/10.

50. The composition according to claim 39, wherein the structuring agent has a tack value ≧0.1 N.s.

51. The composition according claim 39, wherein the structuring agent has a hardness value ≦30 MPa.

52. The composition according to claim 39, comprising from 15 to 50% by weight of structuring agent.

53. The composition according to claim 36, further comprising water, and, optionally, one or more hydrophilic organic solvents.

54. The composition according to claim 53, comprising at least one hydrophilic organic solvent selected from the group consisting of monoalcohols having 2 to 5 carbon atoms, polyols having 2 to 8 carbon atoms, C₃-C₄ ketones, C₂-C₄ aldehydes and mixtures thereof.

55. The composition according to claim 53, wherein the water or the mixture of water and hydrophilic organic solvent is present in an amount ranging from 0.1% to 90% by weight relative to the total weight of the composition.

56. The composition according to claim 36, further comprising a film-forming polymer.

57. The composition according to claim 56, wherein the film-forming polymer is selected from the group consisting of vinyl polymers, polyurethanes, polyesters, polyamides, polyureas, cellulosic polymers, and mixtures thereof.

58. The composition according to claim 56, wherein the film-forming polymer is present in an amount, in terms of dry polymer material, of 0.1% to 60% by weight relative to the total weight of the composition.

59. The composition according to claim 36, further comprising a colorant.

60. The composition according to claim 59, wherein the colorant is selected from the group consisting of pigments, nacres, fat-soluble dyes, water-soluble dyes and mixtures thereof.

61. The composition according to claim 59, wherein the colorant is present in an amount ranging from 0.01% to 30% by weight, relative to the total weight of the composition.

62. The composition according to claim 36, further comprising a cosmetic additive selected from the group consisting of antioxidants, fillers, preservatives, perfumes, neutralizing agents, thickeners, surfactants, cosmetic or dermatological active agents, plasticizers, coalescents and mixtures thereof.

63. The composition according to claim 36, wherein said composition is a makeup, a makeup base, a topcoat composition to be applied over makeup, or a keratin fibre treatment or care composition.

64. The composition according to claim 36, wherein said composition is a composition for coating the eyelashes, a composition to be applied over eyelash makeup, or a composition for treating the eyelashes of human beings or false eyelashes.

65. The composition according to claim 36, wherein said composition is a mascara.

66. A method of treating or making up keratin fibres, which comprises applying to said keratin fibres the composition according to claim 36.

67. A method of coating the eyelashes which comprises applying to the eyelashes the composition according to claim 36.

68. A method of volumizing and separating keratin fibers which comprises applying thereto the composition according to claim 36.
